(19) United States
(12) Patent Application Publication
(10) Pub. No.: US 2011/0008888 A1
(43) Pub. Date: Jan. 13, 2011

(54) Novel Genetic Approaches to Reduce or Inhibit Tumorgenicity of Human Embryonic Stem Cells and Derivatives Following Transplantation

(75) Inventors: Ronald A. Li, Baltimore, MD (US); Tian Xue, Baltimore, MD (US); Chu-Pak Lau, Sacramento, CA (US); Hung-Fat Tse, Sacramento, CA (US)

Correspondence Address: EDWARDS ANGELL PALMER & DODGE LLP, P.O. BOX 55874, BOSTON, MA, 02205, US

(73) Assignee: The John Hopkins University, Baltimore, MD (US)

(21) Appl. No.: 11/884,987

(22) PCT Filed: Feb. 23, 2006
(86) PCT No.: PCT/US06/06661

§ 371 Date: Sep. 17, 2010

Related U.S. Application Data
(60) Provisional application No. 60/655,322, filed on Feb. 23, 2005.

(51) Int. Cl. C12N 5/079; C12N 5/071

(52) U.S. Cl. 435/375

(57) ABSTRACT

Self-renewable embryonic stem cells (ESCs), derived from the inner cell mass of blastocysts, can propagate indefinitely in culture while maintaining their normal karyotypes and pluripotency to differentiate into all cell types. Therefore, ESCs may provide an unlimited supply of even specialized cells such as brain and heart cells for transplantation and cell-based therapies that are otherwise limited by donor availability. However, this promising application is hampered by concerns that ESCs or their multipotent derivatives also possess the potential to form malignant tumors after transplantation in vivo. The present invention provides for a novel genetic method to arrest undesirable cell division (of ESCs and other unwanted lineages) as a means to inhibit or eliminate their tumorgenic potential after transplantation.

### CROSS REFERENCE TO RELATED APPLICATIONS

This application claims priority to U.S. Provisional Patent Applications Ser. No. 60/655,322 filed Feb. 23, 2005, the entire disclosure of which are incorporated herein by reference in its entirety.

### GOVERNMENT SUPPORT

This work was supported by grants from the National Institutes of Health (R01 HL-52768 and R01 HL-72857.

### FIELD OF THE INVENTION

A technology to conditionally arrest cell division of pluripotent human embryonic stem cells (hESCs) and/or their derivatives (or other multipotent stem cells), as a means to inhibit or eliminate their tumorgenic potential after transplantation.

### BACKGROUND OF THE INVENTION

Embryonic stem cells (ESCs) are derived from the inner cell mass of blastocysts. Since ESCs can propagate indefinitely in culture while maintaining their pluripotency to differentiate into all cell types, they may therefore provide an unlimited supply of specialized cells such as cardiomyocytes and neurons for cell-based therapies. For instance, direct injection of pluripotent ESCs after myocardial infarction has been suggested as a means to repair the damaged heart^{1.} However, transplantation of cells with undesirable electrical properties into the heart can predispose patients to lethal electrical disorders (arrhythmias)_{2,3} or that ESCs and their multipotent derivatives also possess the potential to form malignant tumors after transplantation in vivo. Therefore, it is critical to understand the electrophysiological profile of undifferentiated ESCs, which has not been characterized in relation to the physiological function of ion channels in hESC biology, as well as the practical considerations for potential therapeutic applications of hESCs.

Throughout this application, various publications are referenced to by numbers. Full citations for these publications may be found at the end of the specification immediately following the Abstract. The disclosures of these publications in their entireties are hereby incorporated by reference into this application in order to more fully describe the state of the art to those skilled therein as of the date of the invention described and claimed herein.

### SUMMARY OF THE INVENTION

Self-renewable embryonic stem cells (ESCs), derived from the inner cell mass of blastocysts, can propagate indefinitely in culture while maintaining their normal karyotypes and pluripotency to differentiate into all cell types. Therefore, ESCs may provide an unlimited supply of even specialized cells such as brain and heart cells for transplantation and cell-based therapies that are otherwise limited by donor availability. However, this promising application is hampered by concerns that ESCs or their multipotent derivatives also possess the potential to form malignant tumors after transplantation in vivo. The present invention provides for a novel genetic method to arrest undesirable cell division (of ESCs and other unwanted lineages) as a means to inhibit. or eliminate their tumorgenic potential after transplantation. This can be accomplished by genetically and specifically targeting the activity of particular ion channels or proteins that regulate cell division, via ex vivo gene transfer into pluripotent stem cells of specific engineered ion channel (or suicidal) proteins whose expression can be conditionally induced or suppressed (e.g. by the addition of specific ligands, or by a second internal regulatory promoter, etc). The same approaches can be applied to engineer other multipotent stem cells as well as their derivatives to inhibit/prevent their tumorgenic potential.

The present invention provides for the use of undifferentiated, pluripotent ESCs outward K⁺ ion currents whose inhibition arrests cell division or even causes cytotoxicity of ESCs. Therefore, the importance of this finding is to inhibit or even eliminate the tumorgenicity of ESCs by specifically suppressing the current activity of these K⁺ channels in ESCs (and other unwanted lineages) but not in the desired target cell type (e.g. cardiac cells for transplantation into the heart). In brief, our strategy is to overexpress in ESCs an engineered dominant-negative potassium channel construct (e.g. whose pore or active site has been disrupted; see Xue et al 2001 Circ. Res. 90:1267-1273 for an example), or a suicidal gene (e.g. to cause apotosis), under the control of a constitutive promoter whose activity can be conditionally suppressed (e.g. by the addition of a ligand), as well as a second transgene (or siRNA) that suppresses of the activity of our dominant-negative construct or its promoter activity under the control of another promoter that is specific to the target lineage (e.g. myosin heavy chain for cardiac cells).

For culturing of such an engineered ESC line, the expression of our dominant-negative construct (or suicidal gene) will be conditionally suppressed (e.g. in the presence of the appropriate ligand) to enable normal cell division and propagation. Likewise, in vitro differentiation, if needed, will be induced with the dominant-negative transgene (or suicidal gene) similarly suppressed. After transplantation of ESCs (or particular tissue-specific derivatives), the conditional promoter will be turned on (as a result of ligand removal) to arrest cell division of or to cause cytotoxic effect in ESCs (or residual ESCs that contaminate the lineage for transplantation) that can potentially cause tumor. However, the target cell type will remain unaffected because the expression and activity of the chosen dominant-negative construct will be suppressed (by the second transgene or siRNA).

In general, the present invention provides for methods to inhibit or eliminate the tumorgenic potential of pluripotent or multipotent stem cells and/or their derivatives after transplantation and cell-based therapies. An ex vivo approach enables the isolation of clonal genetically-modified cell lines whose transgene location has been characterized to minimize the risk of inappropriate gene insertion (thus, the associated oncogenesis), unlike gene-based approaches.

A preferred embodiment of the present invention provides a method of arresting undesired cell division of pluripotent human embryonic stem cells (ESCs) capable of cell differentiation comprising administering to the stem cells an agent which suppresses potassium current activity in ESCs.

In another embodiment, the invention provides for a method of the suppressing the potassium current activity in stem cells by the transfecting the stem cells with a dominant-negative potassium channel construct with a disrupted pore or active site.

In still other embodiments of the invention, the suppression of potassium current activity in stem cells comprises the transfection of the stem cells with an apoptotic gene under the control of a constitutive promoter that can be conditionally suppressed by the administration of a ligand having such suppressive activity.

One further embodiment of the invention provides that the stem cells are transfected with a second construct whose expression suppresses the activity of the dominant-negative construct. That second construct can be a siRNA polynucleotide which suppresses the activity of the dominant-negative construct.

One other embodiment provides that the stem cells are transfected with a second construct which suppresses the promoter activity of the dominant-negative construct which is under the control of another promoter that is specific to the stem cell once differentiated, such as the promoter for the myosin heavy chain gene for cardiac cells.

The stem cells of the present invention can differentiate into any one of a number of differentiated cells, such as cardiac, neuronal, hepatic, pancreatic cells, etc.

Other aspects of the invention are discussed infra.

### BRIEF DESCRIPTION OF THE FIGURES

FIG. 1 A; Images of pluripotent mESCs immunostained for SSEA-1 and Oct. 4. B; Representative current tracings recorded from undifferentiated mESCs before (left panels) and after blockade by TEA, 4-AP and IBTX (right panels) as indicated; the electrophysiological protocol used for eliciting currents is also given; C) Current-voltage relationship of _{IKDR.} Dose-response relationships for D) TEA and E) 4-AP block of IKDR.

FIG. 2. A) Hyperpolarization-activated currents could be recorded from pluripotent mouse but not human ESCs. Lentivirus-mediated genetic overexpression of HCN1 channels in hESCs led to stable robdust Iₕ expression; B) Steady-state current-voltage relationships; C) Steady-activation curve of Iₕ recorded from stably LV-CAG-HCN1-GFP-transduced hESCs

FIG. 3. A) Expression of ion channel transcripts in mESCs probed by semi-quantitative RT-PCR. Inhibition of proliferation of mESCs by B) TEA C) 4-AP and D) IBTX

FIG. 4. A) Pluripotent hESCs were positive for alkaline phosphatase, SSEA-4 and TRA-1-60; B) Representative current tracings recorded from undifferentiated hESCs; the same electrophysiological from FIG. 1 was used; C) Steady-state current-voltage relationship of _{IKDR} in hESCs. Dose-response relationships for D) TEA block of _{IKDR} and E) TEA inhibition of hESC proliferation.

FIG. 5 A) Microarray anaysis of pluripotent hESCs for the transcript expression of all genes tested using Affemetrix@ U133A microarrays (see Materials and Methods); B) Left: 104 voltage-gated Cav, Nav and Kv channel genes are clustered. The same expression scale bar shown in A) was used. Right: Same as the left panel, except only transcripts that were defined to be expressed, as defined by Affymetrix are shown; C) Bar graph of normalized transcript levels of the expressed ion channel genes. Data were normalized to the expression level of the 50^{th} percentile of the entire microarray.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins.

The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid”, “nucleic acid molecule”, “nucleic acid fragment”, “nucleic acid sequence or segment”, or “polynucleotide” are used interchangeably and may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or RNA molecule or an “isolated” or “purified” polypeptide is a DNA molecule, RNA molecule, or polypeptide that exists apart from its native environment and is therefore not a product of nature. An isolated DNA molecule, RNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, or 5% (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

“Naturally occurring” is used to describe an object that can be found in nature as distinct from being artificially produced. For example, a protein or nucleotide sequence present in an organism (including a virus), which can be isolated from a source in nature and which has not been intentionally modified by a person in the laboratory, is naturally occurring.

The term “chimeric” refers to a gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may include regulatory sequences and coding sequences that are derived from different sources, or include regulatory sequences and coding sequences derived from the same source, but arranged in a manner different from that found in nature.

As used herein, “target gene” refers to a section of a DNA strand of a double-stranded DNA that is complementary to a section of a DNA strand, including all transcribed regions, that serves as a matrix for transcription. A target gene, usually the sense strand, is a gene whose expression is to be selectively inhibited or silenced through RNA interference. As used herein, the term “target gene” specifically encompasses any cellular gene or gene fragment whose expression or activity is associated with the inhibition or prevention of apoptosis. For example, the target gene may be a gene from the Bcl-2 gene family, such as Bcl-2, Bcl-w, and/or Bcl-xL.

A “transgene” refers to a gene that has been introduced into the genome by transformation. Transgenes include, for example, DNA that is either heterologous or homologous to the DNA of a particular cell to be transformed. Additionally, transgenes may include native genes inserted into a non-native organism, or chimeric genes.

The term “endogenous gene” refers to a native gene in its natural location in the genome of an organism.

A “foreign” gene refers to a gene not normally found in the host organism that has been introduced by gene transfer.

The terms “protein,” “peptide” and “polypeptide” are used interchangeably herein.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

“Recombinant DNA molecule” is a combination of DNA sequences that are joined together using recombinant DNA technology and procedures used to join together DNA sequences.

The terms “heterologous gene”, “heterologous DNA sequence”, “exogenous DNA sequence”, “heterologous RNA sequence”, “exogenous RNA sequence” or “heterologous nucleic acid” each refer to a sequence that either originates from a source foreign to the particular host cell, or is from the same source but is modified from its original or native form. Thus, a heterologous gene in a host cell includes a gene that is endogenous to the particular host cell but has been modified through, for example, the use of DNA shuffling. The terms also include non-naturally occurring multiple copies of a naturally occurring DNA or RNA sequence. Thus, the terms refer to a DNA or RNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA or RNA sequence is a sequence that is naturally associated with a host cell into which it is introduced.

“Wild-type” refers to the normal gene or organism found in nature.

“Genome” refers to the complete genetic material of an organism.

A “vector” is defined to include, inter alia, any viral vector, as well as any plasmid, cosmid, phage or binary vector in double or single stranded linear or circular form that may or may not be self transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest but may also code for a functional RNA of interest, for example an antisense RNA, a nontranslated RNA in the sense or antisense direction, or a siRNA. The expression cassette including the nucleotide sequence of interest may be. chimeric. The expression cassette may also be one that is naturally occurring but has been obtained in a recombinant form useful for heterologous expression. The expression of the nucleotide sequence in the expression cassette may be under the control of a constitutive promoter or of an regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence. It may constitute an “uninterrupted coding sequence”, i.e., lacking an intron, such as in a cDNA, or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

The term “open reading frame” (ORF) refers to the sequence between translation initiation and termination codons of a coding sequence. The terms “initiation codon” and “termination codon” refer to a unit of three adjacent nucleotides (a ‘codon’) in a coding sequence that specifies initiation and chain termination, respectively, of protein synthesis (mRNA translation).

“Functional RNA” refers to sense RNA, antisense RNA, ribozyme RNA, siRNA, or other RNA that may not be translated but yet has an effect on at least one cellular process.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters. Examples of promoters that may be used in the present invention include CMV, RSV, polII and polIII promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of siRNA constructs, expression may refer to the transcription of the siRNA only. In addition, expression refers to the transcription and stable accumulation of sense (mRNA) or functional RNA. Expression may also refer to the production of protein.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

“Antisense inhibition” refers to the production of antisense RNA transcripts capable of suppressing the expression of protein from an endogenous gene or a transgene.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” refers to nucleotide sequences that contain one or more regulatory signals, such as one or, more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule. An example of a cis-acting sequence on the replicon is the viral replication origin.

The terms “trans-acting sequence” and “trans-acting element” refer to DNA or RNA sequences whose function does not require them to be on the same molecule.

“Chromosomally-integrated” refers to the integration of a foreign gene or nucleic acid construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix. See http://www.ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions. Generally, stringent conditions are selected to be about 5 degrees C. lower than the thermal melting point (Tm) for the specific sequence at a defined ionic strength and pH. However, stringent conditions encompass temperatures in the range of about 1 degree C. to about 20 degrees C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using a homology alignment algorithm. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tm is the temperature (under defined ionic strength and pH) at which 50% of the target sequence hybridizes to a perfectly matched probe. Specificity is typically the function of post-hybridization washes, the critical factors being the ionic strength and temperature of the final wash solution.

An example of highly stringent wash conditions is 0.15 M NaCl at 72 degrees C. for about 15 minutes. An example of stringent wash conditions is a 0.2×SSC wash at 65 degrees C. for 15 minutes. Often, a high stringency wash is preceded by a low stringency wash to remove background probe signal. An example medium stringency wash for a duplex of, e.g., more than 100 nucleotides, is 1×SSC at 45 degrees C. for 15 minutes. An example low stringency wash for a duplex of, e.g., more than 100 nucleotides, is 4-6×SSC at 40 degrees C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30 degrees C. and at least about 60 degees C. for long probes (e.g., >50 nucleotides). Stringent conditions may also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tm for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37 degrees C., and a wash in 0.1×SSC at 60 to 65degrees C. Exemplary low stringency conditions include hybridization with a buffer solution of 30 to 35% formamide, 1M NaCl, 1% SDS (sodium dodecyl sulfate) at 37 degrees C., and a wash in 1×to 2×SSC (20×SSC=3.0 M NaCl/0.3 M trisodium citrate) at 50 to 55 degrees C. Exemplary moderate stringency conditions include hybridization in 40 to 45% formamide, 1.0 M NaCl, 1% SDS at 37 degrees C., and a wash in 0.5.×to 1×SSC at 55 to 60 degrees C.

Thus, the genes and nucleotide sequences of the invention include both the naturally occurring sequences as well as variant forms. Such variants will continue to possess the desired activity. However, when it is difficult to predict the exact effect of the substitution, deletion, or insertion in advance of doing so, one skilled in the art will appreciate that the effect will be evaluated by routine screening assays.

The term “transformation” or “transfection” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells, and organisms comprising transgenic cells are referred to as “transgenic organisms”.

“Transfected”, “transformed”, “transduced”, “transgenic”, and “recombinant” refer to a host cell or organism into which a heterologous nucleic acid molecule has been introduced. The nucleic acid molecule can be stably integrated into the genome generally known in the art and are disclosed in Sambrook and Russell, infra. See also Innis et al. (1995); and Gelfand (1995); and Innis and Gelfand (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

A subject “infected” with HPV is a subject having cells that contain HPV. The HPV in the cells may not exhibit any other phenotype (i.e., cells infected with HPV do not have to be cancerous). In other words, cells infected with HPV may be pre-cancerous (i.e., not exhibiting any abnormal phenotype, other than those that may be associated with viral infection), or cancerous cells.

An “oncogenic HPV strain” is an HPV strain that is known to cause cervical cancer as determined by the National Cancer Institute (NCI,2001). “Oncogenic E6 proteins” are E6 proteins encoded by the above oncogenic HPV strains. Exemplary oncogenic strains are shown in Table 3. Oncogenic strains of HPV not specifically listed here, are known in the art, and may be found at the world wide website of the National Center for Biotechnology Information (NCBI).

As used herein, the term “derived” or “directed to” with respect to a nucleotide molecule means that the molecule has complementary sequence identity to a particular molecule of interest.

“Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. In some embodiments, gene silencing may be allele-specific. “Allele-specific” gene silencing refers to the specific silencing of one allele of a gene.

“Knock-down,” “knock-down technology” refers to a technique of gene silencing in which the expression of a target gene is reduced as compared to the gene expression prior to the introduction of the siRNA, which can lead to the inhibition of production of the target gene product. The term “reduced” is used herein to indicate that the target gene expression is lowered by 1-100%. For example, the expression may be reduced by 10, 20, 30, 40, 50, 60, 70, 80, 90, 95, or even 99%. Knock-down of gene expression can be directed by the use of dsRNAs or siRNAs. For example, “RNA interference (RNAi),” which can involve the use of siRNA, has been successfully applied to knockdown the expression of specific genes in plants, D. melanogaster, C. elegans, trypanosomes, planaria, hydra, and several vertebrate species including the mouse.

“RNA interference (RNAi)” is the process of sequence-specific, post-transcriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, siRNA induces degradation of target mRNA with consequent sequence-specific inhibition of gene expression.

A “small interfering” or “short interfering RNA” or siRNA is a RNA duplex of nucleotides that is targeted to a gene interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

Furthermore, The term “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). Non limiting examples of siRNA molecules of the invention are shown in FIGS. 18-20, and Table I herein. For example the siRNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siRNA is assembled from a single oligonucleotide, where the self-complementary sense and antisense regions of the siRNA are linked by means of a nucleic acid based or non-nucleic acid-based linker(s). The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siRNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi. The siRNA can also comprise a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiment, the siRNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic intercations, and/or stacking interactions. In certain embodiments, the siRNA molecules of the invention comprise nucleotide sequence that is complementary to nucleotide sequence of a target gene. In another embodiment, the siRNA molecule of the invention interacts with nucleotide sequence of a target gene in a manner that causes inhibition of expression of the target gene. As used herein, siRNA molecules need not be limited to those molecules containing only RNA, but further encompasses chemically-modified nucleotides and non-nucleotides. In certain embodiments, the short interfering nucleic acid molecules of the invention lack 2′ hydroxy (2′-OH) containing nucleotides. Optionally, siRNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

The term “Treating” as used herein refers to ameliorating at least one symptom of, curing and/or preventing the development of a disease or a condition.

### Embryonic Stem Cell Electrophysiology Properties

In the present specification, it is demonstrated that undifferentiated ESCs express several specialized ion channels at the mRNA and functional levels. Although cultured undifferentiated mESCs and hESCs were relatively homogenous when immunostained for pluripotency markers, heterogenous expression of ion channels was observed in mESCs: only fractions of mESCs tested express measurable IK_{DR} and Iₕ. This observation parallels the heterogenous pattern of ion channel expression recently described for human mesenchymal stem cells (hMSCs)^{31.} Unlike mESCs (and hMSCs), however, ion channel expression in hESCs appears to be much more homogenous. I_{KDR} was recorded in all pluripotent hESCs tested (vs. 52.3% of mESCs). Various lines of evidence have suggested that K channels provide a link between physiological and biochemical processes that regulate cell cycle and proliferation by influencing the resting membrane potential (e.g. hyperpolarization is required for the progression of certain cells into the G1 phase of the mitotic cycle) in a number of vastly different cell types from cancer to T-lymphocytes^{11, 32-35}. In accordance with this notion, our results show that pharmacologic blockade of I_{KDR} also inhibits the proliferation of both human and mouse ESCs. In addition to the higher occurrence, the expressed amplitude of I_{KDR} was also ˜10-fold higher in hESCs, which in turn could underlie the more potent effects of K⁺ channel blockers on cell proliferation. Of note, high concentrations of TEA⁺ and IBTX also led to cytotoxic effects. Both the cytotoxicity of K⁺ channel antagonists and their effects on cell proliferation could result from their cellular uptake (e.g. via endocytosis) followed by interactions with some intracellular targets other than K channels. In this regard, K⁺ channel blockers would be anticipated not to affect mESCs that do not express IK_{DR}, if the resultant functional consequences arise solely from their blockade of K⁺ channels.

Not surprisingly, the transcript expression profiles of mESCs and hESCs do not always correspond to the functional expression profile of ion channels. Previously, Van Kempen and colleagues have demonstrated that a variety of ion channel transcripts, such as those of Kv4.3, KvLQT1, Naᵥ, HCN channels, are present in pluripotent mESCs but no ionic currents at all can be detected electrophysiologically before differentiation is induced^{36.} The differences between their observations and those presented here could be attributed to the different mESC lines investigated or the different culturing conditions employed, which may likewise contribute to the “species” differences in the electrophysiological profiles observed between mouse and human ESCs.

Although injection of undifferentiated mESCs into mice does not appear to be arrhythmogenic¹ (unpublished data), the associated electrophysiological consequences could be masked due to the slow current kinetics of IK_{DR} in ESCs relative to the high mouse heart rate (˜600 bpm) and thus extremely short cardiac cycles. Such arrhythmogenic potential could become prominent in species whose heart rates are much slower (e.g. ˜80 bpm for humans). Like MSCs³⁷, pluripotent ESCs also express gap junction proteins²²⁻²⁴ for electrical coupling. Furthermore, ESCs can even subsequently differentiate into electrically-active lineages^{8, 38-40} Taken collectively, our present results highlight additional similarities and differences between mouse and human ESCs, and further suggest that the electrophysiological profile, in addition to the tumorgenic potential, of a given undifferentiated hESC line needs to be carefully assessed before it can be used for therapeutic application, especially when organs or systems where electrical coordination is key for their functions (e.g. cardiac and neuronal) are involved.

As mentioned, pluripotent embryonic stem cells (ESCs) possess promising potential for cell-based therapies but their electrophysiological properties have not been characterized. The presence of ionic currents in mouse (m) and human (h) ESCs and their physiological function is described in the present specification. In mESCs, TEA-sensitive depolarization-activated delayed rectifier K⁺ currents (IK_{DR}; 8.6±0.1 pA/pF at +40 mV; IC₅₀=1.40±0.38 mM), which contained components sensitive to 4-aminopyridine (4-AP; IC₅₀=0.55±0.17 mM) and 100 nM Ca²⁺-activated current (IK_{DR}) blocker iberiotoxin (IBTX), were detected in 52.3% of undifferentiated cells. _{IKDR} was similarly present in hESCs (˜100%) but with a ˜6-fold higher current density (47.5±7.9 pA/pF at +40 mV). Application of TEA, 4-AP or IBTX significantly reduced the proliferation of mESCs and hESCs in a dose-dependent manner (p<0.05). A hyperpolarization-activated inward current (Iₕ; −2.2±0.1 pA/pF at −120 mV) was detected in 23% of mESCs but not hESCs; lentivirus-mediated overexpression of HCN1 channels in hESCs directed stable robust Iₕ expression (−4.5±0.3 pA/pF at −120 mV) without affecting proliferation. Neither Naᵥ nor Caᵥ currents were detected in mESCs and hESCs. Collectively, our results indicate that pluripotent ESCs functionally express a number of specialized ion channels which can be manipulated to affect their potential for undersired cell proliferation.

Examples of preferred administration routes, polynucleotides are provided in the discussion that follows. In general, polynucleotide expression conducive to using the invention is apparent as a shift in a recording (relative to baseline) obtained from at least one of the standard electrophysiological assays. Preferably, administration of a polynucleotide in accordance with the invention provides an increase or decrease of an electrical property by at least about 10% relative to a baseline function. More preferably, the increase or decrease is at least about 20%, more preferably at least about 30% to about 50% or more. That baseline function can be readily ascertained e.g. by performing the electrophysiological assay on a particular mammal prior to conducting the invention methods. Alternatively, related baseline function can be determined by performing a parallel experiment in which a control polynucleotide is administered instead of the polynucleotide of interest. It will be apparent that once a reliable baseline function has been established (or is available from public sources), determination of the baseline function by the practitioner may not always be necessary. Examples of relevant electrical properties are known and include, but are not limited to, at least one of heart rate, refractoriness, speed of conduction, focal automaticity, and spatial excitation pattern.

### Methods for Arresting Undesired Cell Division of Pluripotent ESCs

### Dominant-Negative Constructs

Overexpression of dominant negative potassium channnel construct with an active site that has been disrupted can be used to suppress in ESCs. A dominant negative construct useful in the invention generally contains a portion of the complete potassium channel coding sequence sufficient, for example, for DNA-binding or for a protein-protein interaction such as a homodimeric or heterodimeric protein-protein interaction but lacking the transcriptional activity of the wild type protein. One skilled in the art understands that a dominant negative construct of the potassium channel can be used to suppress expression in a stem cell. A useful dominant negative construct can be a deletion mutant encoding, for example, the active site domain alone or certain intervening regions.

Vectors can be delivered to cells ex vivo, such as embryonic stem cells. Ex vivo cell transfection for diagnostics, research, or for gene therapy (e.g., via re-infusion of the transfected cells into the host organism) is well known to those of skill in the art. In some embodiments, stem cells are isolated from the subject organism, transfected with a nucleic acid, e.g.,, an expression construct expressing an dominant negative construct, an antisense potassium channel nucleic acid (see below), or a ribozyme and the like, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

siRNA

The method of synthesis used for RNA including certain siRNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 .mu.mol scale protocol with a 7.5 min coupling step for alkylsilyl protected nucleotides and a 2.5 min coupling step for 2′-O-methylated nucleotides. Alternatively, syntheses at the 0.2 umol scale can be done on a 96-well plate synthesizer, such as the instrument produced by Protogene (Palo Alto, Calif.) with minimal modification to the cycle. A 33-fold excess (60 uL of 0.11 M=6.6 umol) of 2′-O-methyl phosphoramidite and a 75-fold excess of S-ethyl tetrazole (60 uL of 0.25 M=15 umol) can be used in each coupling cycle of 2′-O-methyl residues relative to polymer-bound 5′-hydroxyl. A 66-fold excess (120 uL of 0.11 M=13.2 umol) of alkylsilyl (ribo) protected phosphoramidite and a 150-fold excess of S-ethyl tetrazole (120 uL of 0.25 M=30 umol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in TBF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65 degrees C. for 10 minutes. After cooling to −20 degrees C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/BF/NMP solution (300 uL of a solution of 1.5 mL N-methylpyrrolidinone, 750 uL TEA and 1 mL TEA.3HF to provide a 1.4 M HF concentration) and heated to 65 degrees C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65 degrees C. for 15 minutes. The vial is brought to room temperature TEA.3HF (0.1 mL) is added and the vial is heated at 65 degrees C. for 15 minutes. The sample is cooled at −20 degrees C. and then quenched with 1.5 M NH₄HCO₃.

For purification of the trityl-on oligomers, the quenched NH₄HCO₃. solution is loaded onto a C-18 containing cartridge that had been prewashed with acetonitrile followed by 50 mM TEAA. After washing the loaded cartridge with water, the RNA is detritylated with 0.5% TFA for 13 minutes. The cartridge is then washed again with water, salt exchanged with 1 M NaCl and washed with water again. The oligonucleotide is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

It will be apparent to one skilled in the art that the inclusion of modified bases, as well as the naturally occuring bases cytosine, uracil, adenosine and guanosine, may confer advantageous properties on siRNA molecules containing said modified bases. For example, modified bases may increase the stability of the siRNA molecule thereby reducing the amount required to produce a desired effect. The provision of modified bases may also provide siRNA molecules which are more or less stable.

### Kits

In another aspect, the invention provides a kit for performing one or a combination of the invention methods disclosed herein. Preferably, the kit includes at least one suitable construct nucleic acid delivery system and preferably at least one desired polynucleotide and/or modified cell containing the genetically modified construct of the potassium channel. Preferably, that polynucleotide is operably linked to the system i.e., it is in functional and/or physical association therewith sufficient to provide for good administration of the polynucleotide into the desired tissue Additionally preferred kits include means for administering the polynucleotide or modified cells containing the genetically modified construct of potassium channel to a mammal such as a syringe, catheter and the like.

All publications and patent applications cited in this specification are herein incorporated by reference as if each individual publication or patent application were specifically and individually indicated to be incorporated by reference.

### EXEMPLIFICATION

### Materials and Methods

### Maintenance of Mouse and Human ESCs

The mESC line R1⁵ (kind gift of Dr. Andras Nagy, University of Toronto) which has been genetically-engineered to constitutively express the green fluorescent protein (GFP) was used in this study to assist their identification from mouse embryonic fibroblast (MEF) cells. mESCs were maintained in their undifferentiated stage by growing on mitomycin-treated MEF feeder layer⁶ in Dulbecco's modified Eagle's medium (DMEM, GIBCO; Carlsbad, Calif.; http://www.lifetech.com) supplemented with 20% fetal bovine serum (Gibco; Carlsbad, Calif.; http://www.lifetech.com), 2 mM L-glutamine, 1 mM sodium pyruvate, 0.1 mM β-mercaptoethanol, 0.1. mM nonessential amino acids and 1000 U/ml leukaemia inhibitory factor (LIF) (Chemicon, Temecula, Calif.; http://www.chemicon.com).

For hESCs, the H1 line (Wicells, Madison, Wis.)⁷ that has been stably transduced by the recombinant lentivirus LV-CAG-GFP (see below for further description of the lentiviral vector employed) to constitutively express GFP under the control of CAG, an internal composite constitutive promoter containing the CMV enhancer and the β-actin promoter, as we have recently described was used⁸. hESCs were maintained on irradiated MEF feeder layer and propagated as previously described. The culture media consisted of DMEM supplemented with 20% fetal bovine serum (HyClone; Logan, Utah, USA; http://www.hvclone.com), 2 mM L-glutamine, 0.1 mM β-mercaptoethanol, and 1% nonessential amino acids. MEF cells were obtained from 13.5 day embryos of CF-1 mice.

Lentivirus-Mediated Stable Genetic Modification of hESCs

For stable genetic modification, we employed the self-inactivating HIV1-based lentiviral vector (LV)^{9.} The plasmid pLV-CAG-HCN1-GFP was created from pRRL-hPGK-GFP SIN-18 (generously provided by Dr. Didier Trono, University of Geneva, Switzerland) by replacing the human phosphoglycerate kinase 1 (hPGK) promoter and the GFP gene with the CAG promoter and the fusion gene HCN1-GFP, whose GFP portion is linked to wild-type (WT) HCN1 at its C-terminus. Transfection of HEK293T cells with pLV-CAG-HCN1-GFP using Lipofectamine 2000 (Invitrogen, Carlsbad, Calif.; http://www.lifetech.com) according to the manufacturer's protocol directed the expression of hyperpolarization-activated currents whose biophysical properties were identical to those of WT HCN1 (data not shown). Recombinant lentiviruses were generated using the 3-plasmid system¹⁰ by co-transfecting HEK293T cells with plenty-CAG-HCN1-GFP, pMD.G and pCMV?R8.91. The latter plasmids encode the vesicular stomatitis virus G envelope protein and the HIV-1 gag/pol, tat, and rev genes required for efficient virus production, respectively. Lentiviral particles were harvested by collecting the culture medium at 48 hours post-transfection, and stored at −80° C. before use.

hESCs were transduced by adding purified lentiviruses to cells at a final concentration of 10,000 TU ml⁻¹ with 8 μg/ml polybrene to facilitate transduction. The multiplicity of infection (MOI) was ˜5 for each round of transduction. After 4 to 6 hours of incubation with LV-CAG-HCN1-GFP, 2 ml fresh medium per 60 mm dish was added. Transduction was allowed to proceed for at least 12-16 hours. Cells were washed with PBS twice to remove residual viral particles. For generating stably LV-CAG-GFP-transduced hESCs, green portions of hESC colonies were microsurgically segregated from the non-green cells, followed by culturing under undifferentiating conditions for expansion. This process was repeated until a homogenous population of green hESC, as confirmed by FACS, was obtained.

### Immunostaining.

Mouse or human ESCs were fixed in 4% paraformaldehyde for 15 min at 21° C., washed with PBS and permeablized with 0.1% Tritton-X-100/PBS. The cells were then blocked with 10% BSA with 0.075% saponin or 4% goat serum in PBS for 2 hours at 21° C. Fixed cells were incubated with the primary antibodies at a dilution of 1:25 (for SSEA-4 and TRA-1-60 in hESCs staining) (Chemicon, Temecula, Calif.; http://www.chemicon.com) overnight at 4° C., followed by incubation with fluorescent-labeled secondary antibodies for 50 minutes at 21° C. and visualization by laser-scanning confocal microscopy.

### Cell Proliferation Assay

Specified concentrations of TEA, 4-AP or IBTX were added to mESCs or hESCs, followed by counting trypsinized viable cells with a hemocytometer after 48 hours, or measuring colony parameters under microscopy after 6 days (due to the slower growth rate of human cells), respectively, as previously performed for studying the action of K channel blockers on cell proliferation¹¹.

### Electrophysiology

Only GFP-expressing cells were selected for experiments. Electrophysiological recordings were performed at room temperature using whole-cell patch clamp. Pipette electrodes (TW120E-6; World Precision Instruments, Sarasota, Fla.; http://www.wpiinc.com) were fabricated using a Sutter P-87 horizontal puller, fire-polished, and had final tip resistances of 2 to 4 MO. All recordings were performed at room temperature in a bath solution containing (in mM): 110 NaCl, 30 KCl, 1.8 CaCl₂, 0.5 MgCl₂, 5 HEPES, 10 glucose, pH adjusted to 7.4 with NaOH. The internal solution for patch recordings contained (in mM): 10 NaCl, 130 KCl, 0.5 MgCl₂, 5 HEPES, 1 EGTA, 5 MgATP, pH adjusted to 7.3 with KOH. The voltage dependence of Iₕ activation was assessed by plotting tail currents measured immediately after pulsing to −140 mV as a function of the preceding 3-second test pulse normalized to the maximum tail current recorded. Data were fit to the Boltzmann function using the Marquardt-Levenberg algorithm in a nonlinear least-squares procedure: mₛ=1/{1+exp[(V₁-V_{1/2})/k]}, where V₁ is the test potential, V_{1/2} is the half-point of the relationship, and k=RT/zF is the slope factor. Half-blocking concentrations (IC₅₀) were determined from the following binding isotherm: I/Iₒ=1/{1+([blocker]/IC₅₀)} where _{IC50} is the half-blocking concentration, Io and I are the peak currents measured at the voltage indicated before and after application of the blocker, respectively.

### RT-PCR

Total RNA was prepared from mESCs and mouse brain using ToTALLY RNA™ Kit (Ambion Inc., Tex.; http://www.ambion.com). Single stranded cDNA was synthesized from ˜1 μg of total RNA using random hexamers and SuperScript™ reverse transcriptase (Invitrogen, Carlsbad, Calif.; http://www.lifetech.com) according to the manufacturer's protocols, followed by PCR amplification with gene-specific primers for ion channel genes. Primers, annealing temperatures, product sizes and the corresponding references are given in Table 1.18S ribosomal RNA (498 bp) was used as an internal control. The reaction was conducted using the following protocol: Initial denaturing of the template for 5 minutes at 94° C. followed by 32 repeating cycles of denaturing for 1 minute at 94° C., annealing for 1 minute, extension for 1 minute at 72° C. and a final elongation at 72° C. for 7 minutes. The PCR products were size-fractionated by 1% agarose gel electrophoresis and visualized by ethidium bromide staining.

### Microarray Analysis

Microarray analysis was performed using Affymetrix human genome U133A array, which represents 18,400 transcript and variants, including 14,500 well-characterized human genes. Total RNA was extracted from pluripotent human ESCs (H1) and hybridized to microarrays according to the protocols provided by the manufacturer. The software Genespring 6.0 (Silicon Genetics, Redwood City, Calif.; http://www.silicongenetics.com) was used for microarray data analysis. Data were normalized to the expression level of the 50^{th} percentile of the entire chip and filtered to show genes that are labeled as expressed (i.e. ‘present’ flags) as defined by Affymetrix analysis.

### Statistics

All data reported are means ±S.E.M. Statistical significance was determined for all individual data points and fitting parameters using one-way ANOVA and Tukey's HSD post hoc test at the 5% level.

### EXAMPLES

### Example 1

### Ionic Currents in Pluripotent Mouse ESCs

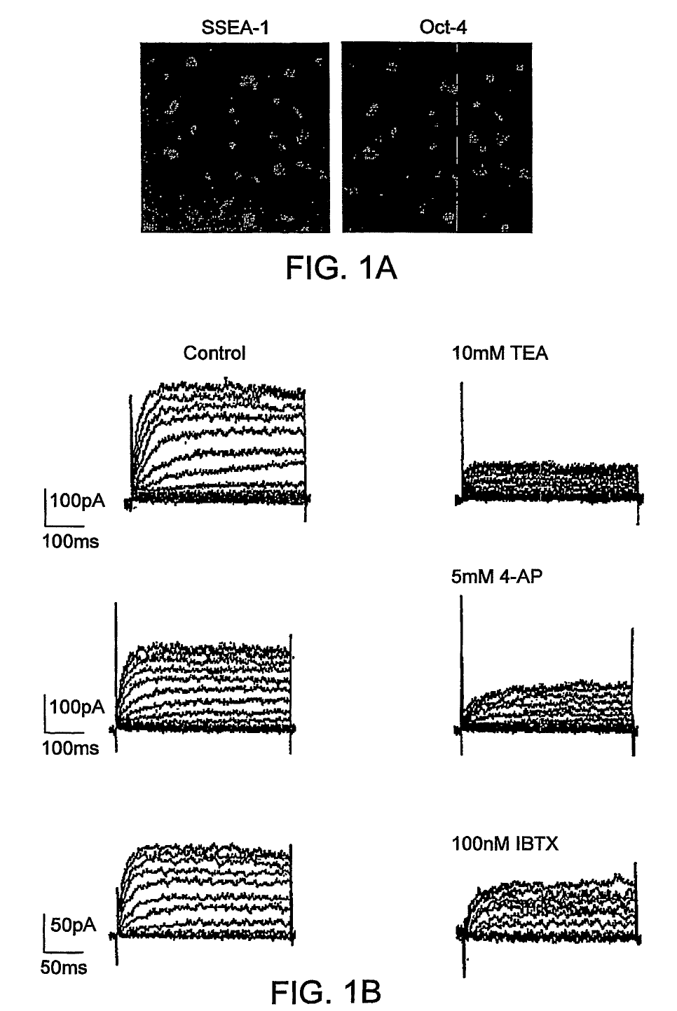

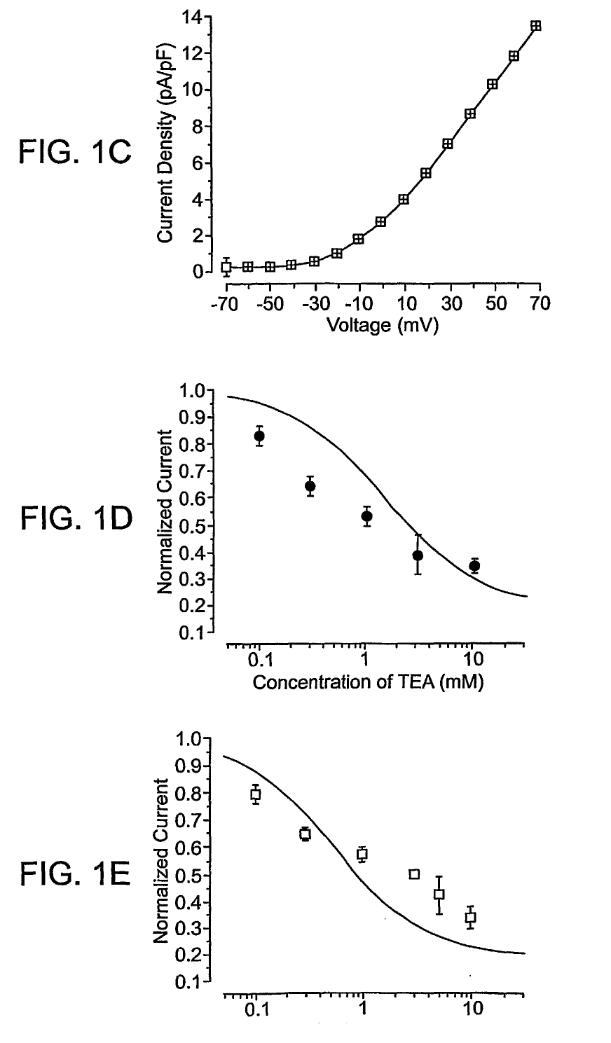

FIG. 1A shows that undifferentiated mESC colonies were homogenously immunostained for the pluripotency markers Oct4 and SSEA-1^{12, 13}. In 159 of 304 (52.3%) undifferentiated mESCs, depolarization-activated time-dependent non-inactivating outward currents that increased progressively with positive voltages could be recorded (8.6±0.1 pA/pF at +40 mV; FIG. 1B-C). These outwardly rectifying currents resemble the delayed rectifier K⁺ currents (IK_{DR}), and could be dose-dependently inhibited by the known ^{K+} channel blocker TEA^{+14, 15} (IC₅₀=1.40±0.38 mM, n=12; FIG. 1B, D). IK_{DR} in mESCs was also sensitive to 4-aminopyridine (4-AP; IC₅₀=0.55±0.17 mM, n=7), a more potent K channel blocker than TEA^{+16, 17} and the Ca²⁺-activated large-conductance K⁺ current (IK_{Ca}) blocker iberiotoxin (IBTX; current inhibition=33.2±12.7%, n=3) (FIG. 1B, E). Although voltage-gated Na⁺ (Naᵥ) and Ca²⁺ (Caᵥ) currents were completely absent in all pluripotent mESCs tested (n>200), a modest yet detectable hyperpolarization-activated inward current (a.k.a. Iₕ, encoded by the hyperpolarization-activated cyclic nucleotide-modulated non-selective or HCN ion channel family¹⁸; −2.2±0.04 pA/pF at −120 mV) was detected in 79 of 270 cells (29.3%). Iₕ in mESCs was reversibly blocked by the HCN inhibitor Cs+19, 20. Inwardly rectifying K currents I_{K1} responsible for stabilizing the resting membrane potential were also not present.

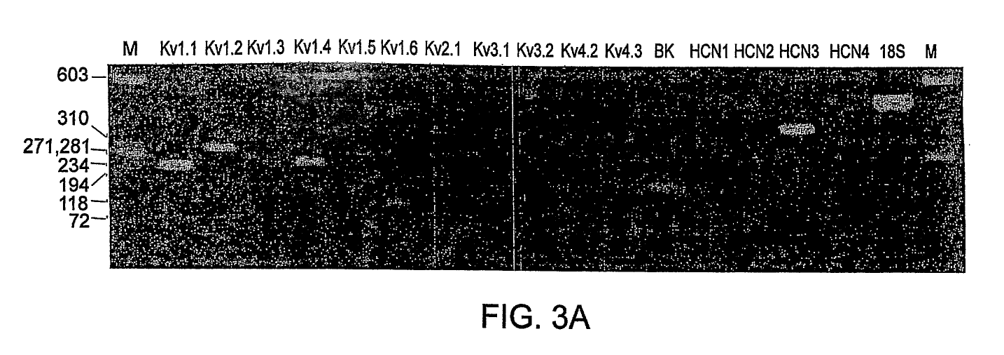

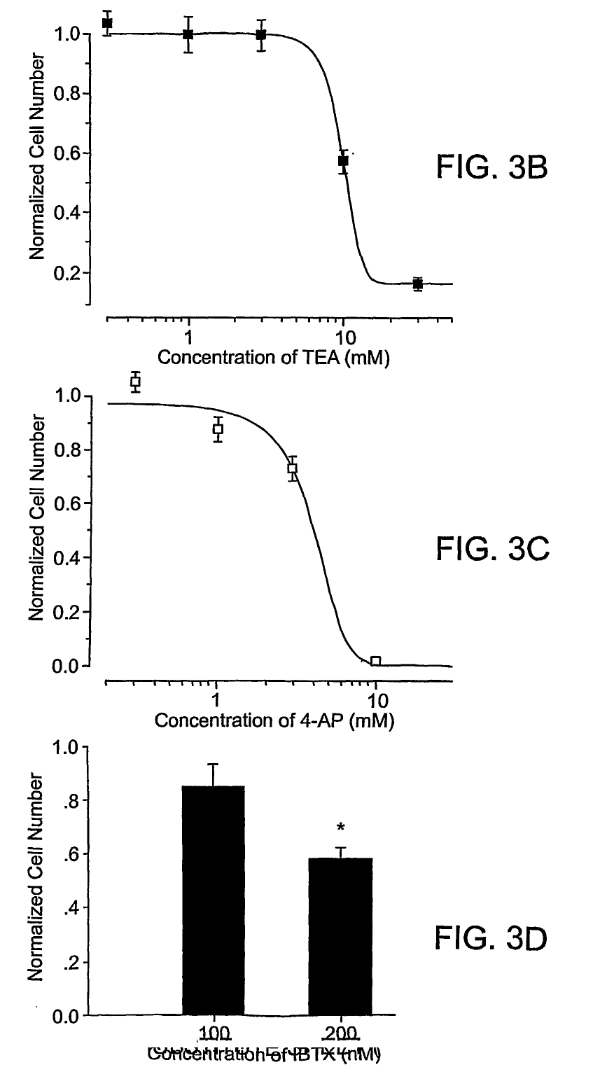

To obtain insights into the molecular identities of the ionic currents identified, total RNA was isolated from pluripotent mESCs for RT-PCR. FIG. 3A shows that Kv1.1, 1.2, 1.3, 1.4, 1.6, 4.3, and BK (or Maxi-K) transcripts but not Kv2.1, 3.1, 3.2 and 4.3 were detected. Consistent with the presence of Iₕ, HCN2 and HCN3 transcripts were also expressed. While Kv1.1, 1.2, 1.6 and BK channels might underline the delayed rectifier current recorded, Kv1.4- and 4.3-encoded transient outward K⁺ currents (Iₗₒ) were not observed electrophysiologically.

### Example 2

### Effects of Ion Channel Blockers

To investigate possible physiological roles of the ionic currents identified, we next studied the functional consequences of their pharmacological blockade. Application of TEA+ significantly inhibited the proliferation of mESCs in a dose-dependent manner (FIG. 3B) when cell number was assessed as previously described^{11.} The half-effective concentration (EC₅₀) was 11.9±1.2 mM (n=8), approximately ˜10-fold higher than the IC₅₀^{per}_{IKDR} inhibition. Similarly, 4-AP (EC₅₀=4.0±1.8 mM; FIG. 3C) and IBTX

(FIG. 3D) also dose-dependently reduced cell proliferation. Of note, the rank orders of these agents to inhibit proliferation generally follow the trend of their potencies to block IK_{DR} (i.e. 4-AP>TEA).

### Example 3

### Electrophysiological Properties of Human ESCs: Similarities and Differences

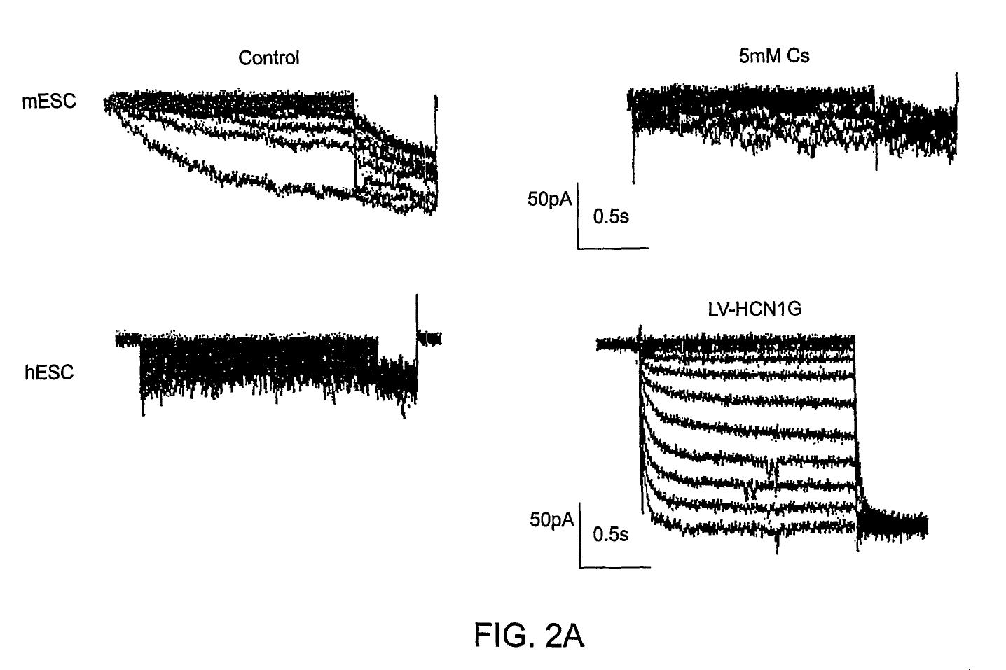

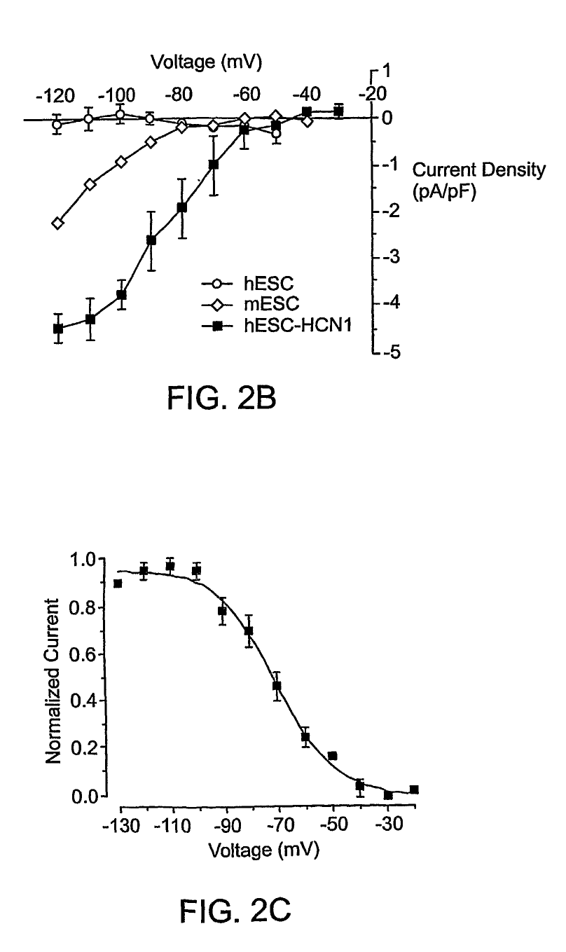

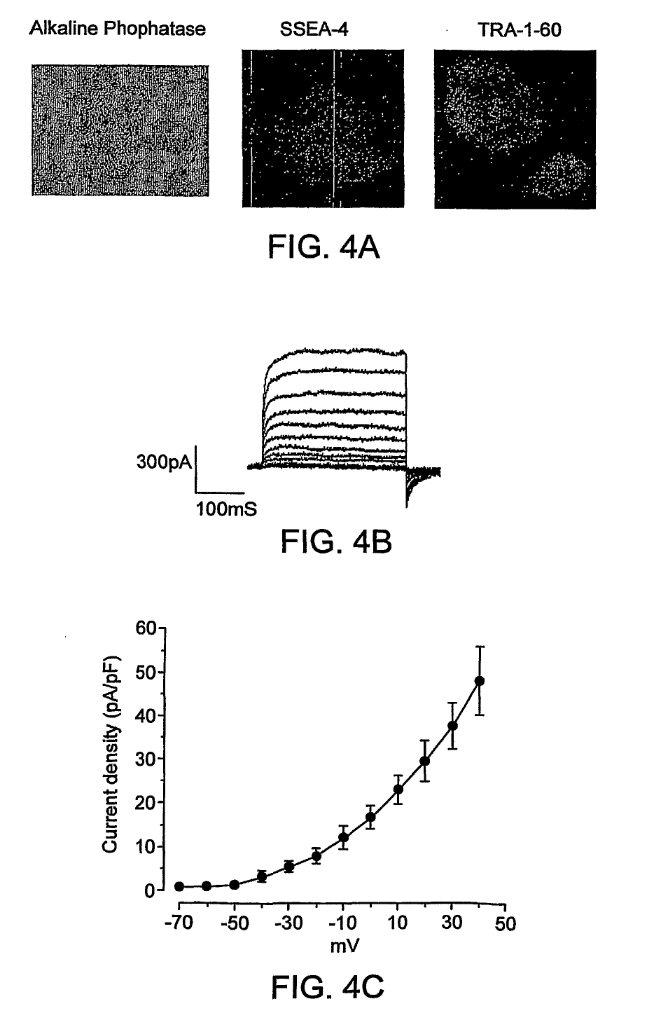

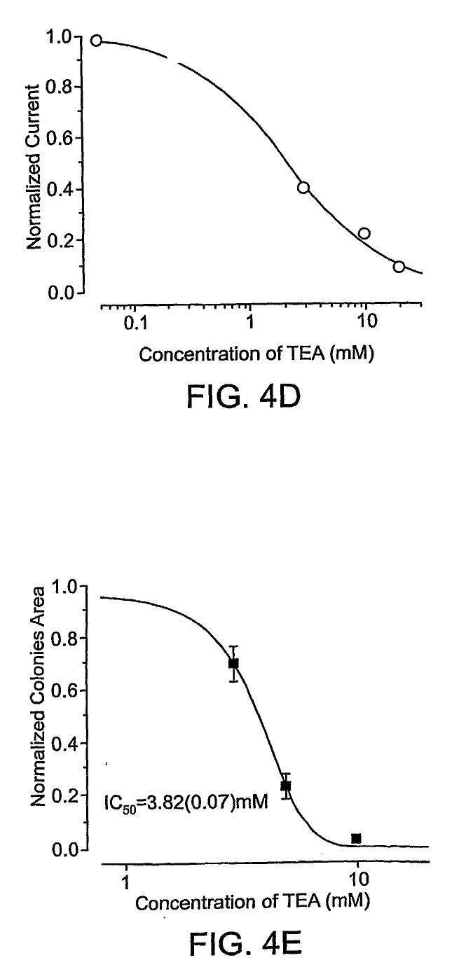

Although hESCs and mESCs share a number of similarities, significant differences are known to exist between the two species^{21.} Therefore, we also examined the previously unexplored electrophysiological properties of hESCs. Pluripotent hESCs were positive for markers such as alkaline phosphatase, Oct4, SSEA4, and TRA-60 (FIG. 4A), consistent with previous reports⁷. Similar to mESCs, TEA⁺-sensitive IK_{DR} (IC₅₀=2.1±0.2 mM) was also detected in hESCs (˜100%) but the current density was ˜6-fold higher (47.5±7.9 pA/pF at +40 mV, n=12, p<0.05) (FIG. 4B-C). Application of TEA⁺ dose-dependently inhibited hESC proliferation with an EC₅₀ (3.8±0.1 mM) similar to the IC₅₀ for current blockade (FIG. 4D-E). Unlike mESCs, however, there was no measurable Iₕ in all hESCs tested (n=30; FIG. 2A). Recently, Iₕ has been reported to express in electrically-active hESC-derived cardiac derivatives²²⁻²⁴. To explore a possible role of Iₕ, we overexpressed the molecular correlate HCN channels in hESCs. Lentivirus-mediated genetic overexpression of HCN1 channels in hESCs led to stable robust Iₕ expression (−4.5±0.3 pA/pF at −120 mV; FIG. 2A, C) without affecting IK_{DR}, cell viability and proliferation (data not shown). These results suggest that unlike IK_{DR}, Iₕ does not influence cell proliferation. Same as mESCs, neither Naᵥ nor Caᵥ currents could be detected in hESCs.

### Example 4

### Microarray Analysis of Ion Channel Genes in hESCs

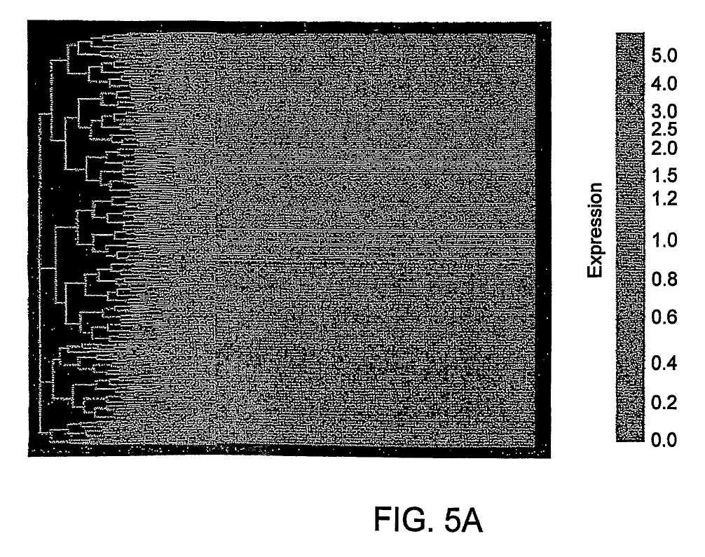

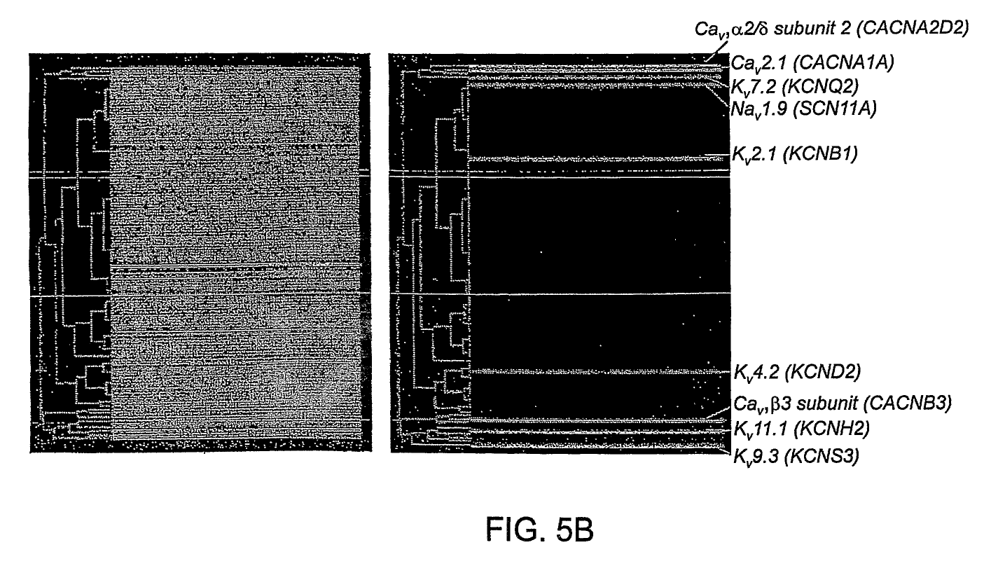

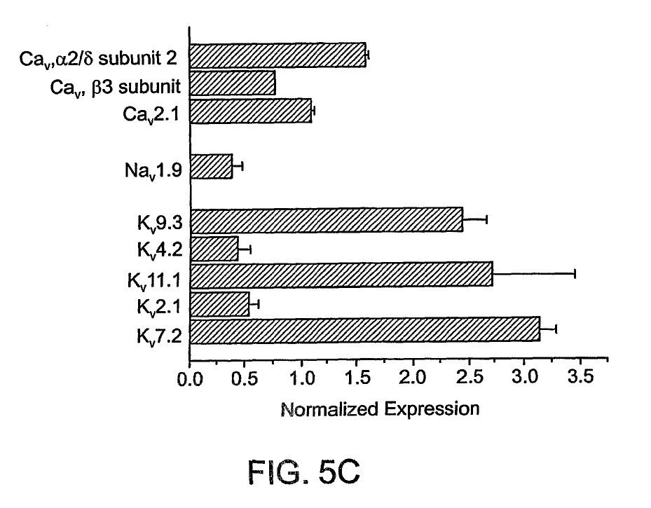

Using Affemetrix@ U133A chips, we performed microarray anaysis of pluirpotent hESCs to examine the expression of ion channels at the transcriptomic level. FIG. 5A shows the expression profile of all genes tested in undifferentiated hESCs. For voltage-gated ion channels, a total of 36, 19 and 49 genes on the U133A chips were identified as Caᵥ, Naᵥ, or Kᵥ channel genes, respectively. For inspection, their expression profile was extracted from FIG. 5A, and further summarized in FIGS. 5B-C by normalizing signals to the average expression level of the entire microarray in a manner similar to that of cytokines and their receptors in hESCs as recently reported by Dvash et al ^{25.} Our data indicate that among the total 104 voltage-gated ion channel genes mentioned above, only the transcripts of 3, 1 and 5 Ca, Naᵥ and Kᵥ genes were significantly expressed, as defined by Affymetrix. The corresponding gene products were CACNA1A (Caᵥ2.1), CACNA2D2 (Caᵥ a2/d subunit 2), CACNB3 (Caᵥ β3 subunit), SCN11A (Naᵥ1.9), KCNB1 (Kᵥ2.1), KCND2 (Kᵥ4.2), KCNQ2 (Kᵥ7.2), KCNS3 (Kᵥ9.3), and KCNH2 (Kᵥ11.1) (note that although I_{Ca}, I_{Na} and Kv4.2-encoded transient outward K⁺ currents could not be electrophysiologically recorded, like mESCs). Of note, KCNQ2 and KCNH2, which underlie the non-inactivating, slowly deactivating M-current²⁶ and the rapid component of the cardiac delayed rectifier (IK)^{27,} were relatively highly expressed. Similarly, KCNB1 and KCNS3, which encode for the delay rectifier Kᵥ2.1 channels and the silent modulatory a-subunit Kᵥ9.3 that heteromerizes with Kᵥ2.1 subunits²⁸⁻³⁰, respectively, were also expressed. Collectively, these ion channel genes could underlie the K_{DR} current identified, although further experiments will be needed to confirm and dissect their molecular identities. By contrast, no HCN transcript was expressed in pluripotent hESCs.

TABLE 1

| | | | | | Annealing | |
|---|---|---|---|---|---|---|
| | | Forward primer | Reverse primer | Length | temperature | |
| Gene | Acc. -No. | sequence (5′-3′) | sequence (5′-3′) | (bp) | (?) | Reference |
| HCN1 | NM_010408 | CTCTTTTTGCTAACGCCGAT | CATTGAAATTGTCCACCGAA | 291 | 57 | Proc Natl Acad Sci |
| | | | | | | USA. 2003; 100: |
| | | | | | | 15235-15240. |
| HCN2 | NM_008226 | GTGGAGCGAGCTCTACTCGT | GTTCACAATCTCCTCACGCA | 229 | 57 | Proc Natl Acad Sci |
| | | | | | | USA. 2003; 100: |
| | | | | | | 15235-15240. |
| HCN3 | NM_008227 | GACACCCGCCTCACTGATGGAT | GTTTCCGCTGCAGTATCGAATTC | 370 | 57 | Proc Natl Acad Sci |
| | | | | | | USA. 2003; 100: |
| | | | | | | 15235-15240. |
| HCN4 | XM_287905 | TGCTGTGCATTGGGTATGGA | TTTCGGCAGTTAAAGTTGATG | 337 | 47 | Proc Natl Acad Sci |
| | | | | | | USA. 2003; 100: |
| | | | | | | 15235-15240. |
| Kv1.1 | NM_010595 | GCCTCTGACAGTGACCTCAGC | GGGACAGGAGTCGCCAAGGG | 240 | 57 | Glia. 1997; 20: |
| | | | | | | 127-134. |
| Kv1.2 | NM_008417 | CGTCCTCCCCTGACCTAAA | CCATGCAGAACCAGATGCTGTAG | 296 | 57 | Glia. 1997; 20: |
| | | | | | | 127-134. |
| Kv1.3 | NM_008418 | ATCTTCAAGCTCTCCCGCCA | CGATCACCATATACTCCGAC | 478 | 53 | Am J Physiol Cell |
| | | | | | | Physiol. 2000; |
| | | | | | | 279: C1123-1134. |
| Kv1.4 | NM_021275 | CTCCTCCCATGATCCTCAAGG | GCAGGTCTGTGTACGAACACC | 257 | 57 | Glia. 1997; 20: |
| | | | | | | 127-134. |
| Kv1.5 | NM_145983 | GCCATTGCCATCGTGTCGGT | ACATGTGGTCTCCACGATGA | 242 | 53 | Am J Physiol Cell |
| | | | | | | Physiol. 2000; |
| | | | | | | 279: C1123-1134. |
| Kv1.6 | NM_013568 | GCTTGGCAAACCTGACTTTGC | CCTGTTTTCCTGCAGGCC | 136 | 57 | Glia. 1997; 20: |
| | | | | | | 127-134. |
| Kv2.1 | NM_008420 | CGGCAGTTCAACCTGATCCC | TTTATTGCCCAGAATGCTGTCG | 468 | 57 | Biochem Biophys |
| | | | | | | Res Con 2004; |
| | | | | | | 313: 156-162. |
| Kv3.1 | NM_008421 | CGAGCTGGAGATGACCAAG | AAGAAGAGGGAGGCAAAGG | 156 | 60 | Designed with |
| | | | | | | Primer Prerr |
| Kv3.2 | U52223 | AATAGCCATGCCTGTGC | AGCGTCTGATAGGGAGC | 296 | 60 | Designed with |
| | | | | | | Primer prerr. |
| Kv4.2 | NM_019697 | ATCGCCCATCAAGTCACAGTC | CCGACACATTGGCATTAGGAA | 111 | 53 | J Physiol. 2002; |
| | | | | | | 544: 403-41 |
| Kv4.3 | NM_019931 | CAAGACCACCTCACTCATCGA | TCGAGCTCTCCATGCAGTTCT | 176 | 60 | J Physiol. 2002; |
| | | | | | | 544: 403-41 |
| BK | NM_010610 | CCATTAAGTCGGGCTGATTTAAG | CCTTGGGAATTAGCCTGCAAGA | 188 | 53 | J Biol Chem. |
| | | | | | | 2003; 278:453 |

### REFERENCES

The following specific references, also incorporated by reference, are indicated in the examples and discussion above by a corresponding reference number.

1. Hodgson D M, Behfar A, Zingman L V et al. Stable benefit of embryonic stem cell therapy in myocardial infarction. Am J Physiol Heart Circ Physiol. 2004;287:H471-479.

2. Menasche P, Hagege A A, Vilquin J T et al. Autologous skeletal myoblast transplantation for severe postinfarction left ventricular dysfunction. J Am Coll Cardiol. 2003;41:1078-1083.

3. Smits P C, van Geuns R J, Poldermans D et al. Catheter-based intramyocardial injection of autologous skeletal myoblasts as a primary treatment of ischemic heart failure: Clinical experience with six-month follow-up. J Am Coll Cardiol. 2003;42:2063-2069.

4. Wang K, Xue T, Tsang S Y et al. Electrophysiological properties of pluripotent human and mouse embryonic stem cells. Circulation. 2004;Scientific Sessions Abstracts Supplement.

5. Nagy A, Rossant J, Nagy Ret al. Derivation of completely cell culture-derived mice from early-passage embryonic stem cells. Proc Natl Acad Sci USA. 1993;90:8424-8428.

6. Doetschman T C, Eistetter H, Katz Metal. The in vitro development of blastocyst-derived embryonic stem cell lines: Formation of visceral yolk sac, blood islands and myocardium. J Embryol Exp Morphol. 1985;87:27-45.

7. Thomson J A, Itskovitz-Eldor J, Shapiro S S et al. Embryonic stem cell lines derived from human blastocysts. Science. Vol 282; 1998:1145-1147.

8. Xue T, Cho H, Akar F et al. Genetically-engineered human embryonic stem cell cardiac derivatives as surrogate biological pacemakers. Circulation. 2004.

9. Trono D. Lentiviral vectors. New York: Spring-Verlag Berlin Heidelberg; 2002.

10. Zufferey R, Dull T, Mandel R J et al. Self-inactivating lentivirus vector for safe and efficient in vivo gene delivery. J. Virol. Vol 72; 1998:9873-9880.

11. Vaur S, Bresson-Bepoldin L, Dufy B et al. Potassium channel inhibition reduces cell proliferation in the gh3 pituitary cell line. J Cell Physiol. 1998;177:402-410.

12. Solter D, Knowles B B. Monoclonal antibody defining a stage-specific mouse embryonic antigen (ssea-1). Proc Nail Acad Sci USA. 1978;75:5565-5569.

13. Niwa H, Miyazaki J, Smith A G. Quantitative expression of oct-3/4 defines differentiation, dedifferentiation or self-renewal of es cells. Nat Genet. 2000;24:372-376.

14. Hille B. The selective inhibition of delayed potassium currents in nerve by tetraethylammonium ion. J. Gen. Physiol. 1967;50:1287-1302.

15. Kirsch G E, Taglialatela M, Brown A M. Internal and external tea block in single cloned k+ channels. Am J Physiol. 1991;261:C583-590.

16. Mathie A, Wooltorton J R A, Watkins C S. Voltage-activated potassium channels in mammalian neurons and their block by novel pharmacological agents. General Pharmacology: The Vascular System. 1998;30:13-24.

17. Baker M, Howe J R, Ritchie J M. Two types of 4-aminopyridine-sensitive potassium current in rabbit schwann cells. J Physiol. 1993;464:321-342.

18. Pape H-C. Queer current and pacemaker: The hyperpolarization-activated cation current in neurons. Annual Review of Physiology. 1996;58:299-327.

19. Champigny G, Bois P, Lenfant J. Characterization of the ionic mechanism responsible for the hyperpolarization-activated current in frog sinus venosus. Pflugers Arch. 1987;410:159-164.

20. McCormick D A, Pape H C. Properties of a hyperpolarization-activated cation current and its role in rhythmic oscillation in thalamic relay neurones. J. Physiol. 1990;431:291-318.

21. Boheler K R, Czyz J, Tweedie D et al. Differentiation of pluripotent embryonic stem cells into cardiomyocytes. Circ Res. Vol 91; 2002:189-201.

22. Satin J, Kehat I, Caspi O et al. Mechanism of spontaneous excitability in human embryonic stem cell derived cardiomyocytes. J Physiol (Lond). 2004jphysiol.2004.068213.

23. Sohl G, Theis M, Hallas Get al. A new alternatively spliced transcript of the mouse connexin32 gene is expressed in embryonic stem cells, oocytes, and liver. Exp Cell Res. 2001;266:177-186.

24. Oyamada Y, Komatsu K, Kimura H et al. Differential regulation of gap junction protein (connexin) genes during cardiomyocytic differentiation of mouse embryonic stem cells in vitro. Exp Cell Res. 1996;229:318-326.

25. Dvash T, Mayshar Y, Darr H et al. Temporal gene expression during differentiation of human embryonic stem cells and embryoid bodies. Hum Reprod. 2004.

26. Cooper E C, Jan L Y. M-channels: Neurological diseases, neuromodulation, and drug development. Arch Neurol. 2003;60:496-500.

27. Pond A L, Nerbonne J M. Erg proteins and functional cardiac i(kr) channels in rat, mouse, and human heart. Trends Cardiovasc Med. 2001;11:286-294.

28. Kerschensteiner D, Monje F, Stocker M. Structural determinants of the regulation of the voltage-gated potassium channel kv2.1 by the modulatory alpha-subunit kv9.3. J Biol Chem. 2003;278:18154-18161.

29. Kerschensteiner D, Stocker M. Heteromeric assembly of kv2.1 with kv9.3: Effect on the state dependence of inactivation. Biophys J. 1999;77:248-257.

30. Stocker M, Kerschensteiner D. Cloning and tissue distribution of two new potassium channel alpha-subunits from rat brain. Biochem Biophys Res Commun. 1998;248:927-934.

31. Heubach J F, Graf E M, Leutheuser J et al. Electrophysiological properties of human mesenchymal stem cells. J Physiol. 2004;554:659-672.

32. Parihar A S, Coghlan M J, Gopalakrishnan M et al. Effects of intermediate-conductance ca2+-activated k+ channel modulators on human prostate cancer cell proliferation. Eur J Phannacol. 2003;471:157-164.

33. Wonderlin W F, Strobl J S. Potassium channels, proliferation and gl progression. J Membr Biol. 1996;154:91-107.

34. Platoshyn O, Golovina V A, Bailey C L et al. Sustained membrane depolarization and pulmonary artery smooth muscle cell proliferation. Am J Physiol Cell Physiol. 2000;279:C1540-1549.

35. Vautier F, Belachew S, Chittajallu Ret al. Shaker-type potassium channel subunits differentially control oligodendrocyte progenitor proliferation. Glia. 2004.

36. van Kempen M J A, van Ginneken A, de Grijs I et al. Expression of the electrophysiological system during murine embryonic stem cell cardiac differentiation. Cell Physiol and Biochem. 2003;13:263-270.

37. Valiunas V, Doronin S, Valiuniene Let al. Human mesenchymal stem cells make cardiac connexins and form functional gap junctions. J Physiol (Lond). 2004;555:617-626.

38. Kehat I, Kenyagin-Karsenti D, Snir Metal. Human embryonic stem cells can differentiate into myocytes with structural and functional properties of cardiomyocytes. J Clin Invest. 2001;108:407-414.

39. Mummery C, Ward-van Oostwaard D, Doevendans P et al. Differentiation of human embryonic stem cells to cardiomyocytes: Role of coculture with visceral endoderm-like cells. Circulation. 2003;107:2733-2740.

40. He J Q, Ma Y, Lee Yet al. Human embryonic stem cells develop into multiple types of cardiac myocytes: Action potential characterization. Circ Res. 2003;93:32-39.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

We claim:

1. A method of arresting undesired cell division of pluripotent human embryonic stem cells (ESCs) capable of cell differentiation comprising administering to the stem cells an agent which suppresses potassium current activity in ESCs.

2. A method of claim 1 wherein the suppression of potassium current activity in stem cells comprises the transfection of the stem cells with a dominant-negative potassium channel construct with a disrupted pore or active site.

3. A method of claim 1 wherein the suppression of potassium current activity in stem cells comprises the transfection of the stem cells with an apoptotic gene under the control of a constitutive promoter can be conditionally suppressed.

4. A method of claim 3 wherein the promoter activity can be conditionally suppressed by the administration of a ligand that suppresses the activity of the constitutive promoter.

5. A method of claim 2 wherein the stem cells are transfected with a second construct whose expression suppresses the activity of the dominant-negative construct.

6. A method of claim 2 wherein the stem cells are transfected with a second construct for a siRNA polynucleotide which suppresses the activity of the dominant-negative construct.

7. A method of claim 2 wherein the stem cells are transfected with a second construct which suppresses the promoter activity of the dominant-negative construct which is under the control of another promoter that is specific to the stem cell once differentiated.

8. A method of claim 7 wherein the other promoter which is specific to the stem cell once differentiated is the promoter for the myosin heavy chain gene for cardiac cells.

9. A method of claim 1 wherein the stem cells differentiate into cardiac cells.

10. A method of claim 1 wherein the stem cells differentiate into neuronal cells.

11. A method of claim 1 wherein the stem cells differentiate into hepatic cells.

12. A method of claim 1 wherein the stem cells differentiate into pancreatic cells.
